(19) United States
(12) Patent Application Publication
(10) Pub. No.: US 2025/0107702 A1
(43) Pub. Date: Apr. 3, 2025

(54) OPTICAL UNIT, ENDOSCOPE, AND METHOD OF ASSEMBLING OPTICAL UNIT

(71) Applicant: OLYMPUS MEDICAL SYSTEMS CORP., Tokyo (JP)

(72) Inventor: Toru KUCHIMARU, Hachioji-shi (JP)

(73) Assignee: OLYMPUS MEDICAL SYSTEMS CORP., Tokyo (JP)

(21) Appl. No.: 18/899,693

(22) Filed: Sep. 27, 2024

Related U.S. Application Data
(60) Provisional application No. 63/586,701, filed on Sep. 29, 2023.

(51) Int. Cl. A61B 1/00

(52) U.S. Cl. CPC A61B 1/00197

(57) ABSTRACT

An optical unit capable of curbing penetration of moisture into the optical unit is provided. The optical unit includes a plurality of lenses constituting an optical system, a cylindrical first lens frame that holds at least a first lens disposed closest to an object side among the plurality of lenses and has a first screw portion formed thereon, and a cylindrical holding frame on which a second screw portion is formed, wherein the first screw portion and the second screw portion are fitted to each other such that the first lens frame is fixed to the holding frame.

The present invention relates to an optical unit, an endoscope, and a method of assembling the optical unit. Priority is claimed on U.S. Provisional Application No. 63/586,701, filed Sep. 29, 2023, the content of which is incorporated herein by reference.

### BACKGROUND OF THE INVENTION

### Field of the Invention

### Description of Related Art

Conventionally, an endoscope equipped with an optical unit configured to fix an objective unit that holds an objective lens to a holder is known (for example, Patent Document 1).

### PATENT DOCUMENTS

[Patent Document 1] International Publication No. 2015/136064

### SUMMARY OF THE INVENTION

Among a plurality of lenses included in the optical unit, the objective lens is easily affected by the external temperature, and thus if moisture penetrates into the optical unit, the objective lens is prone to condensation. Therefore, in order to curb condensation on the objective lens, it is necessary to curb penetration of moisture into the optical unit. In the optical unit, the gap between the objective unit and the holder is sealed by an adhesive that fixes the objective unit and the holder. This can curb penetration of moisture into the optical unit to some extent. However, in cases where the optical unit is washed with water and a cleaning solution, and the like, for example, there is a risk that moisture will penetrate into the optical unit through the adhesive.

In view of the above circumstances, an object of the present invention is to provide an optical unit, an endoscope, and a method of assembling the optical unit that can prevent moisture from entering the interior of the optical unit.

An optical unit in one aspect of the present invention to achieve the aforementioned object is an optical unit provided at the tip of an endoscope, including a plurality of lenses constituting an optical system, a cylindrical first lens frame that holds at least a first lens disposed closest to an object side among the plurality of lenses and has a first screw portion formed thereon, and a cylindrical holding frame on which a second screw portion is formed, wherein the first screw portion and the second screw portion are fitted to each other such that the first lens frame is fixed to the holding frame.

An endoscope in one aspect of the present invention has the optical unit provided at the tip thereof.

A method of assembling an optical unit in one aspect of the present invention is a method of assembling an optical unit to be provided at a tip of an endoscope, the method including an adjustment process of adjusting the position of a first lens disposed closest to an object side among a plurality of lenses constituting an optical system and held by a first lens frame, and a fixing process of fixing the first lens frame to a holding frame, wherein the position of the first lens is adjusted while a first screw portion formed on the first lens frame is screwed into a second screw portion formed on the holding frame, and then the first lens frame is fixed to the holding frame.

According to the present invention, it is possible to provide an optical unit, an endoscope, and a method of assembling the optical unit that can prevent moisture from entering the inside of the optical unit.

### BRIEF DESCRIPTION OF THE DRAWINGS

FIG. 1 is a perspective view showing an endoscope system of a first embodiment.

FIG. 2 is a longitudinal cross-sectional view showing an optical unit of the first embodiment.

FIG. 3 is an enlarged longitudinal cross-sectional view showing the optical unit of the first embodiment.

FIG. 4 is a flowchart showing a method of assembling an optical unit of the first embodiment.

FIG. 5 is a first longitudinal cross-sectional view showing the method of assembling an optical unit of the first embodiment.

FIG. 6 is a second longitudinal cross-sectional view showing the method of assembling an optical unit of the first embodiment.

FIG. 7 is a third longitudinal cross-sectional view showing the method of assembling an optical unit of the first embodiment.

FIG. 8 is an enlarged longitudinal cross-sectional view showing an optical unit of a second embodiment.

FIG. 9 is an enlarged longitudinal cross-sectional view showing an optical unit of a modified example of the second embodiment.

FIG. 10 is an enlarged longitudinal cross-sectional view showing an optical unit of a third embodiment.

FIG. 11 is an enlarged longitudinal cross-sectional view showing an optical unit of a fourth embodiment.

### DETAILED DESCRIPTION OF THE INVENTION

Hereinafter, an optical unit, an endoscope, and a method of assembling the optical unit according to an embodiment of the present invention will be described with reference to the drawings. Note that the scope of the present invention is not limited to the following embodiment and can be modified as desired within the scope of the technical concept of the present invention. In addition, in the following drawings, the scale and number of each structure may differ from the actual structure in order to make each configuration easier to understand.

In each figure, the Z axis is shown as appropriate. The Z axis is a direction in which an optical axis J of an embodiment described below extends. The optical axis J shown as appropriate in each figure is a virtual axis. In the following description, the direction in which the optical axis J extends, that is, the direction parallel to the Z axis, is called an “optical axis direction.” The radial direction having the optical axis J as a center is simply called a “radial direction.” The circumferential direction having the optical axis J as a center is simply called a “circumferential direction.” The side in the optical axis direction toward which a Z-axis arrow points (+Z side) is an “object side (distal side),” and the opposite side in the optical axis direction toward which the Z-axis arrow points (−Z side) is an “image side (proximal side).” The circumferential direction is indicated by an arrow θ in each figure.

### First Embodiment

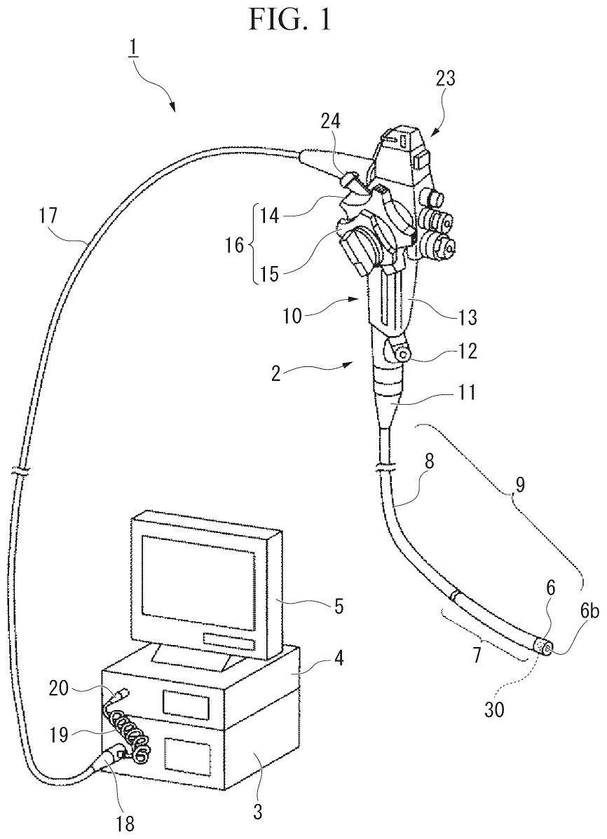

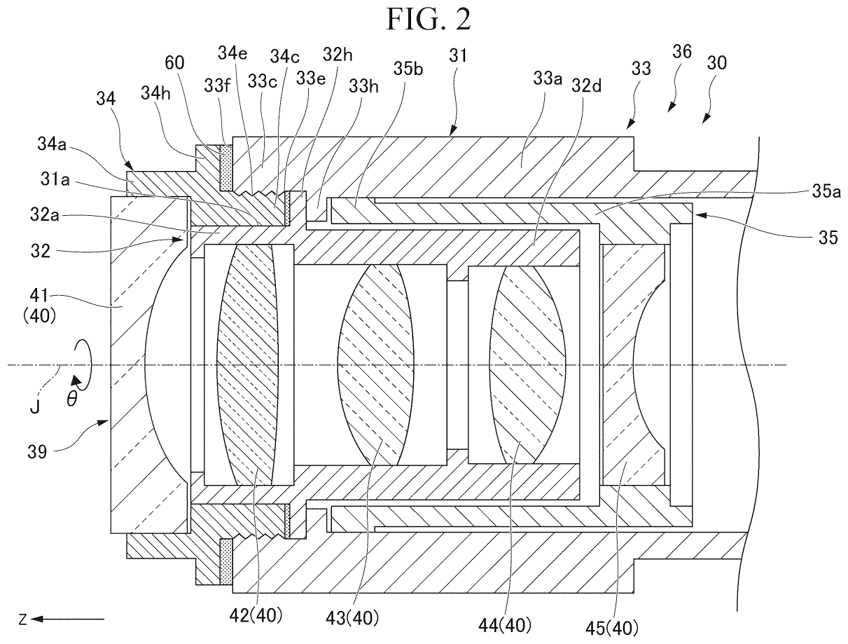

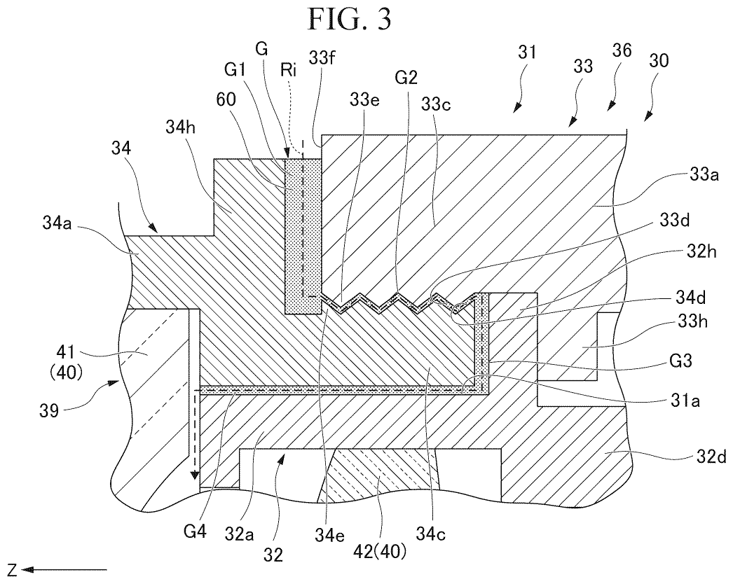

FIG. 1 is a perspective view showing an endoscope system 1 of the present embodiment. FIG. 2 is a longitudinal cross-sectional view showing an optical unit 30 of the present embodiment. FIG. 3 is an enlarged longitudinal cross-sectional view showing the optical unit 30 of the present embodiment.

The endoscope system 1 shown in FIG. 1 includes an endoscope 2, a light source device 3, a video processor 4, and a color monitor 5. The use of the endoscope 2 of the present embodiment is not particularly limited, and may be, for example, a medical endoscope or an industrial endoscope. The endoscope 2 includes an insertion part 9 inserted into a subject, an operation part 10 disposed outside the subject, and a universal cord 17 extending from the operation part 10.

A light guide bundle that is not shown and an electric cable that forms an electrical signal transmission path are inserted into the universal cord 17. A scope connector 18 that optically couples illumination light from the light source device 3 that supplies the illumination light to the incident end of the light guide bundle is provided at the rear end of the universal cord 17. The electric cable inside the universal cord 17 extends to the inside of a scope cable 19 that branches off from the scope connector 18. An electric connector part 20 that electrically connects the end of the electric cable that is not shown inside the scope cable 19 to the video processor 4 is provided at the end of the scope cable 19. When the electric connector part 20 is connected to the video processor 4, electrical components built into the endoscope 2 and the video processor 4 are connected such that they can communicate through the electric cable.

The insertion part 9 includes a tip portion 6, a bending portion 7, and a flexible tube portion 8 in this order from the distal end, which is the front end of the direction of insertion into the subject, toward the proximal end that connects to the operation part 10. A well-known tip opening that is not shown, an observation window 6b, and an illumination window that is not shown are formed on the tip surface of the tip portion 6. An optical unit 30 for imaging the subject in front of the tip portion 6 is disposed at a position facing the observation window 6b inside the tip portion 6. That is, the optical unit 30 is provided at the tip of the endoscope 2. A detailed configuration of the optical unit 30 will be described later. A distal end of the light guide bundle for transmitting illumination light is disposed on the rear side of the illumination window. The light guide bundle is inserted into the insertion part 9, passes through the inside of the operation part 10, and extends into the inside of the universal cord 17. A proximal end of the light guide bundle is located inside the scope connector 18. When the scope connector 18 is connected to the light source device 3, illumination light from the light source device 3 is optically coupled to the proximal end of the light guide bundle. As a result, illumination light generated by the light source device 3 is optically transmitted to the illumination window in the tip portion 6, and is emitted from the illumination window to the outside of the tip portion 6.

The bending portion 7 is connected to the proximal end of the tip portion 6. The bending portion 7 is bendable and tubular. By operating the operation part 10, the bending amount and bending direction of the bending portion 7 can be changed, and thus the orientation of the tip portion 6 can be changed. The bending portion 7 includes, for example, a plurality of nodal rings. The nodal rings are rotatably connected to adjacent nodal rings. In the bending portion 7, two types of operation wires including a first operation wire and a second operation wire, the light guide bundle, the electric cable, a treatment tool channel which will be described later, and the like are inserted inside the nodal rings. Each operation wire extends from the bending portion 7 through the inside of the flexible tube portion 8 to the operation part 10.

The operation part 10 includes an operation part main body 13 that is held by a user and is used for various operations of the endoscope 2. A bending operation part 16, a switch part 23, and an operation lever 24 are provided on the upper part of the operation part main body 13. The bending operation part 16 has operation knobs 14 and 15 that operate the bending amount and bending direction of the bending portion 7. The operation knobs 14 and 15 can independently pull the first operation wire and the second operation wire.

The switch part 23 includes at least one switch that is mainly used to operate an imaging function. The operation lever 24 is used to operate the adjustment amount of each of a focus adjustment function, a magnification adjustment function, and a zoom magnification function of the optical unit 30 which will be described later. A forceps port 12 and a folding stopper 11 are provided at the bottom of the operation part main body 13. The forceps port 12 is an opening that communicates with the proximal end of the treatment tool channel that extends from the inside of the operation part 10 through the insertion part 9 to the tip opening of the tip portion 6. The folding stopper 11 connects the proximal end of the insertion part 9 and the operation part 10.

As described above, the optical unit 30 is provided at the tip of the endoscope 2. The optical unit 30 forms an optical image of a subject. As shown in FIG. 2, the optical unit 30 is cylindrical and extends in the optical axis direction. The optical unit 30 includes a housing 36, a plurality of lenses 40, and an adhesive 60. The housing 36 is a case that holds the plurality of lenses 40. The housing 36 includes a holding frame 31, a first lens frame 34, and a moving frame 35.

The holding frame 31 is cylindrical and extends in the optical axis direction. The holding frame 31 holds the first lens frame 34, the moving frame 35, and some of the plurality of lenses 40. The holding frame 31 includes a first holding frame 32 and a second holding frame 33. A recess 31a is provided in the holding frame 31.

The first holding frame 32 is substantially cylindrical and extends in the optical axis direction with the optical axis J as a center. The first holding frame 32 includes a first tubular portion 32a, a second tubular portion 32d, and a first annular portion 32h. The first tubular portion 32a is substantially cylindrical and extends in the optical axis direction with the optical axis J as a center. The end of the first tubular portion 32a on the object side (+Z side) is the end of the first holding frame 32 on the object side.

The second tubular portion 32d is cylindrical and extends in the optical axis direction with the optical axis J as a center. The second tubular portion 32d is connected to the end of the first tubular portion 32a on the image side (−Z side) in the optical axis direction. The end of the second tubular portion 32d on the image side is the end of the first holding frame 32 on the image side. In the present embodiment, the inner diameter and the outer diameter of the second tubular portion 32d are less than the inner diameter and the outer diameter of the first tubular portion 32a. The first annular portion 32h is an annular plate that protrudes outward in the radial direction from the connection portion of the first tubular portion 32a and the second tubular portion 32d. The plate surface of the first annular portion 32h faces the optical axis direction.

The second holding frame 33 holds the first holding frame 32, the first lens frame 34, and the moving frame 35. The second holding frame 33 is substantially cylindrical and extends in the optical axis direction with the optical axis J as a center. The second holding frame 33 surrounds the first holding frame 32 and the moving frame 35 from the outside in the radial direction. The second holding frame 33 includes a third tubular portion 33a, a fourth tubular portion 33c, and a second annular portion 33h.

The third tubular portion 33a is substantially cylindrical and extends in the optical axis direction with the optical axis J as a center. The end of the third tubular portion 33a on the image side (−Z side) is the end of the second holding frame 33 on the image side. The third tubular portion 33a surrounds the second tubular portion 32d and the moving frame 35 from the outside in the radial direction. The third tubular portion 33a is disposed having a gap from the second tubular portion 32d in the radial direction. The end of the third tubular portion 33a on the object side (+Z side) is located closer to the image side than the first annular portion 32h.

The fourth tubular portion 33c is substantially cylindrical and extends in the optical axis direction with the optical axis J as a center. The end of the fourth tubular portion 33c on the object side (+Z side) is the end of the second holding frame 33 on the object side. The end of the fourth tubular portion 33c on the image side (−Z side) is connected to the third tubular portion 33a in the optical axis direction. The fourth tubular portion 33c surrounds the first tubular portion 32a and the first annular portion 32h from the outside in the radial direction. The fourth tubular portion 33c is disposed having a gap from the first tubular portion 32a in the radial direction. The outer diameter of the fourth tubular portion 33c is the same as the outer diameter of the third tubular portion 33a. The inner diameter of the fourth tubular portion 33c is greater than the inner diameter of the third tubular portion 33a. The fourth tubular portion 33c has a first opposing surface 33f. As shown in FIG. 3, the fourth tubular portion 33c has a first inner surface 33d.

The first opposing surface 33f is the surface of the outer surface of the fourth tubular portion 33c that faces the object side (+Z side). When viewed from the optical axis direction, the first opposing surface 33f is a substantially annular shape with the optical axis J as a center. The first inner surface 33d is the inner circumferential surface of the fourth tubular portion 33c, that is, the surface that faces inward in the radial direction. In the present embodiment, the first annular portion 32h is fitted in the portion of the first inner surface 33d that faces the image side (−Z side). Accordingly, the first holding frame 32 is fixed to the second holding frame 33. The first holding frame 32 and the second holding frame 33 may be fixed by an adhesive.

A second screw portion 33e is formed on the fourth tubular portion 33c. That is, the second screw portion 33e is formed on the holding frame 31. The second screw portion 33e is a female screw formed on the first inner surface 33d. That is, the second screw portion 33e is formed on the inner circumferential surface of the holding frame 31. The second screw portion 33e is formed on a portion of the first inner surface 33d that is closer to the object side (+Z side) than the first annular portion 32h.

As shown in FIG. 2, the second annular portion 33h is an annular plate that protrudes inward in the radial direction from the connection portion of the third tubular portion 33a and the fourth tubular portion 33c. The plate surface of the second annular portion 33h faces the optical axis direction. The second annular portion 33h is located closer to the image side (−Z side) than the first annular portion 32h. The second annular portion 33h contacts the first annular portion 32h in the optical axis direction and supports the first annular portion 32h in the optical axis direction. This determines the position of the first holding frame 32 in the optical axis direction relative to the second holding frame 33.

As shown in FIG. 3, the recess 31a is recessed from the first opposing surface 33f toward the image side (−Z side). In the present embodiment, the recess 31a is composed of the first tubular portion 32a, the first annular portion 32h, and the fourth tubular portion 33c. The inner surface of the recess 31a is composed of the outer circumferential surface of the first tubular portion 32a, the surface of the first annular portion 32h facing the object side (+Z side), and the first inner surface 33d. The second screw portion 33e is formed on the surface of the recess 31a facing inward in the radial direction. The recess 31a extends around the entire circumference. When viewed from the optical axis direction, the recess 31a is a substantially annular depression.

As shown in FIG. 2, the first lens frame 34 holds at least a first lens 41, which will be described later, among the plurality of lenses 40. The first lens frame 34 may hold lenses other than the first lens 41. The first lens frame 34 is tubular and extends in the optical axis direction. In the present embodiment, the first lens frame 34 is substantially cylindrical with the optical axis J as a center. The first lens frame 34 includes a holding portion 34a, a protrusion portion 34c, and a flange portion 34h. In the present embodiment, the holding portion 34a, the protrusion portion 34c, and the flange portion 34h are each part of the same single member. The holding portion 34a, the protrusion portion 34c, and the flange portion 34h may be different members. In this case, the first lens frame 34 can be configured by fixing the holding portion 34a, the protrusion portion 34c, and the flange portion 34h to each other, for example, using an adhesive. The holding portion 34a is cylindrical and protrudes in the optical axis direction with the optical axis J as a center. The end of the holding portion 34a on the object side (+Z side) is the end of the first lens frame 34 on the object side.

The flange portion 34h is a substantially circular ring shape with the optical axis J as a center. The flange portion 34h is connected to the end of the holding portion 34a on the image side (−Z side). The outer diameter of the flange portion 34h is greater than the outer diameter of the holding portion 34a. The outer portion of the flange portion 34h in the radial direction protrudes outward in the radial direction from the holding portion 34a. The inner diameter of the flange portion 34h is less than the inner diameter of the holding portion 34a. The flange portion 34h is disposed closer to the object side (+Z side) than the second holding frame 33. The outer portion of the flange portion 34h in the radial direction is disposed having a gap from the first opposing surface 33f in the optical axis direction. The flange portion 34h faces the first opposing surface 33f in the optical axis direction.

The protrusion portion 34c is cylindrical and protrudes from the inner portion of the flange portion 34h toward the image side (−Z side) in the radial direction. In the present embodiment, the protrusion portion 34c is a substantially cylindrical shape with the optical axis J as a center. The portion of protrusion portion 34c on the image side is disposed inside recess 31a. That is, at least a part of the protrusion portion 34c is disposed inside recess 31a.

As shown in FIG. 3, the protrusion portion 34c has a first outer surface 34d. The first outer surface 34d is the outer circumferential surface of the protrusion portion 34c, that is, the surface facing outward in the radial direction. The first outer surface 34d faces the first inner surface 33d in the radial direction. The surface of the protrusion portion 34c facing inward in the radial direction faces the outer circumferential surface of the first tubular portion 32a in the radial direction. The surface of the protrusion portion 34c facing inward in the radial direction faces the surface of the recess 31a facing outward in the radial direction having a gap in the radial direction. The protrusion portion 34c faces the first annular portion 32h having a gap in the optical axis direction.

The protrusion portion 34c has a first screw portion 34e formed thereon. That is, the first screw portion 34e is formed on the first lens frame 34. The first screw portion 34e is formed on the first outer surface 34d. The first screw portion 34e is a male screw formed on the outer circumferential surface of the first lens frame 34. The first screw portion 34e is disposed closer to the image side (−Z side) than the flange portion 34h. That is, the flange portion 34h is disposed closer to the object side (+Z side) than the first screw portion 34e. The first screw portion 34e is fitted to the second screw portion 33e. The first lens frame 34 is fixed to the holding frame 31 by fitting the first screw portion 34e and the second screw portion 33e to each other. In the present embodiment, a gap is provided between the first screw portion 34e and the second screw portion 33e. The first screw portion 34e and the second screw portion 33e may contact each other.

In the present embodiment, a gap G is provided between the holding frame 31 and the first lens frame 34. The gap G connects the outside of the optical unit 30 and the inside of the optical unit 30. The gap G includes a first gap G1, a second gap G2, a third gap G3, and a fourth gap G4. The first gap G1 is a gap between the first opposing surface 33f and the flange portion 34h. The first gap G1 connects the outside of the optical unit 30 and the second gap G2. The first gap G1 extends in the radial direction. The second gap G2 is a gap between the first screw portion 34e and the second screw portion 33e. The second gap G2 connects the first gap G1 and the third gap G3. In a vertical cross-sectional view, the second gap G2 has a sawtooth shape extending in the optical axis direction. Therefore, in the present embodiment, the length of the second gap G2 can be made longer compared to a case in which the first outer surface 34d and the first inner surface 33d are not provided with the first screw portion 34e and the second screw portion 33e respectively, that is, the first outer surface 34d and the first inner surface 33d are flat surfaces facing the radial direction.

The third gap G3 is a gap between the protrusion portion 34c and the first annular portion 32h. The third gap G3 connects the second gap G2 and the fourth gap G4. The third gap G3 extends in the radial direction. The fourth gap G4 is a gap between the surface of the protrusion portion 34c facing inward in the radial direction and the surface of the recess 31a facing outward in the radial direction. The fourth gap G4 connects the third gap G3 and the inside of the optical unit 30. The fourth gap G4 extends in the optical axis direction.

A penetration path Ri shown in FIG. 3 is a path through which moisture from outside the optical unit 30 penetrates into the optical unit 30. In the present embodiment, the penetration path Ri is the gap G. As described above, in the present embodiment, since the first screw portion 34e and the second screw portion 33e are formed on the first outer surface 34d and the first inner surface 33d, the length of the second gap G2 can be increased. This makes it possible to lengthen the penetration path Ri.

As shown in FIG. 2, the moving frame 35 has a substantially cylindrical shape extending in the optical axis direction with the optical axis J as a center. The moving frame 35 is disposed inside the second holding frame 33. The moving frame 35 includes a moving main body portion 35a and a moving protrusion portion 35b. The moving main body portion 35a has a substantially cylindrical shape extending in the optical axis direction with the optical axis J as a center. The portion of the moving main body portion 35a on the object side (+Z side) is disposed between the second tubular portion 32d and the third tubular portion 33a. The moving main body portion 35a is disposed having gap from the second tubular portion 32d and the third tubular portion 33a in the radial direction. The moving protrusion portion 35b protrudes outward in the radial direction from the end of the moving main body portion 35a on the object side. The moving protrusion portion 35b is annular with the optical axis J as a center. The moving protrusion portion 35b contacts the inner circumferential surface of the third tubular portion 33a in the radial direction. As a result, the moving frame 35 is held by the second holding frame 33 such that the moving frame 35 is movable in the optical axis direction relative to the second holding frame 33.

The plurality of lenses 40 constitute an optical system 39 of the optical unit 30. The optical system 39 forms an optical image of a subject. The plurality of lenses 40 include the first lens 41, a second lens 42, a third lens 43, a fourth lens 44, and a fifth lens 45. The lenses are disposed from the object side (+Z side) to the image side (−Z side) in the order of the first lens 41, the second lens 42, the third lens 43, the fourth lens 44, and the fifth lens 45. The configuration and the like of the optical system 39 is not limited to the present embodiment, and for example, the number of lenses constituting the optical system 39 may be four or less, or six or more.

The first lens 41 is a lens disposed closest to the object side (+Z side) among the plurality of lenses 40. The first lens 41 is an objective lens. The first lens 41 is held on the inner circumferential surface of the holding portion 34a. That is, the first lens frame 34 holds at least the first lens 41. The first lens 41 is exposed to the outside of the housing 36. The lenses 42, 43, 44, and 45 other than the first lens 41 among the plurality of lenses 40 are housed inside the housing 36. Therefore, in the present embodiment, the first lens 41 is a lens that is most susceptible to the temperature outside the optical unit 30 among the plurality of lenses 40. In the following description, the lenses 42, 43, 44, and 45 other than the first lens 41 among the plurality of lenses 40 may be referred to as the other lenses 42 to 45.

The second lens 42 is held on the inner circumferential surface of the first tubular portion 32a. The third lens 43 and the fourth lens 44 are held on the inner circumferential surface of the second tubular portion 32d. As a result, the holding frame 31 holds at least one of the lenses disposed closer to the image side (−Z side) than the first lens 41 among the plurality of lenses 40. The fifth lens 45 is held on the inner circumferential surface of the moving main body portion 35a. The fifth lens 45 is movable in the optical axis direction along with the moving frame 35 relative to the second holding frame 33. In the present embodiment, the focal position of the optical image of the subject in the optical axis direction can be adjusted by moving the moving frame 35 in the optical axis direction.

The adhesive 60 fixes the holding frame 31 and the first lens frame 34. As shown in FIG. 3, the adhesive 60 is filled in the gap G. The adhesive 60 is filled in each of the first gap G1, the second gap G2, the third gap G3, and the fourth gap G4. That is, the adhesive 60 is filled in the first gap G1, that is, between the flange portion 34h and the first opposing surface 33f. The adhesive 60 is filled in the second gap G2, that is, between the first screw portion 34e and the second screw portion 33e. The adhesive 60 is filled in the third gap G3, that is, between the protrusion portion 34c and the first annular portion 32h. The adhesive 60 is filled in the fourth gap G4, that is, between the surface of the protrusion portion 34c facing inward in the radial direction and the surface of the recess 31a facing outward in the radial direction. As a result, the gap G between the holding frame 31 and the first lens frame 34 is sealed by the adhesive 60. In the present embodiment, as the adhesive 60, for example, a thermosetting adhesive such as an epoxy resin adhesive, a melamine resin adhesive, or a phenol resin adhesive can be used.

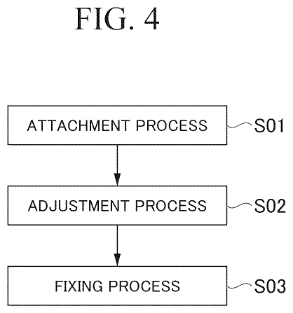

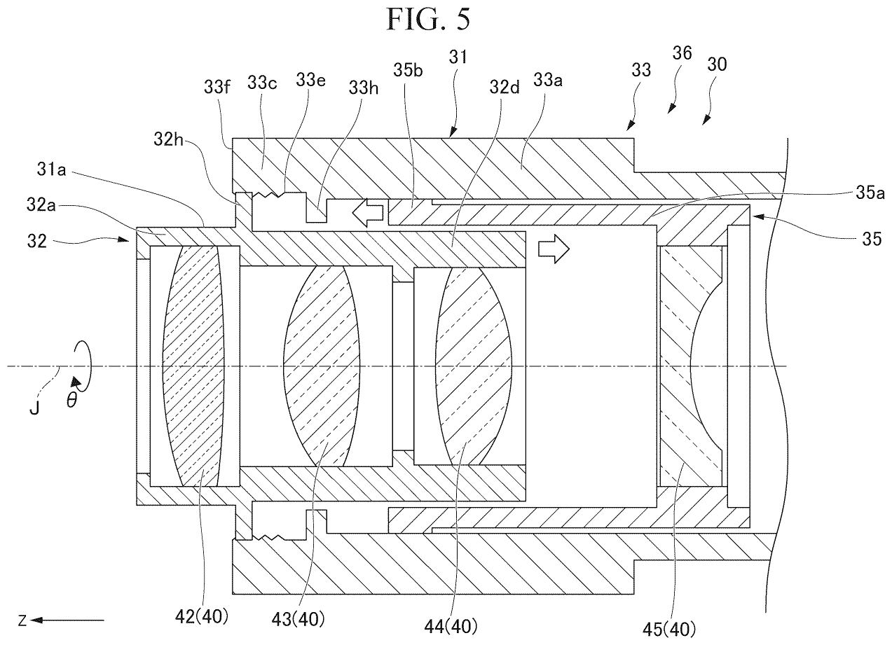

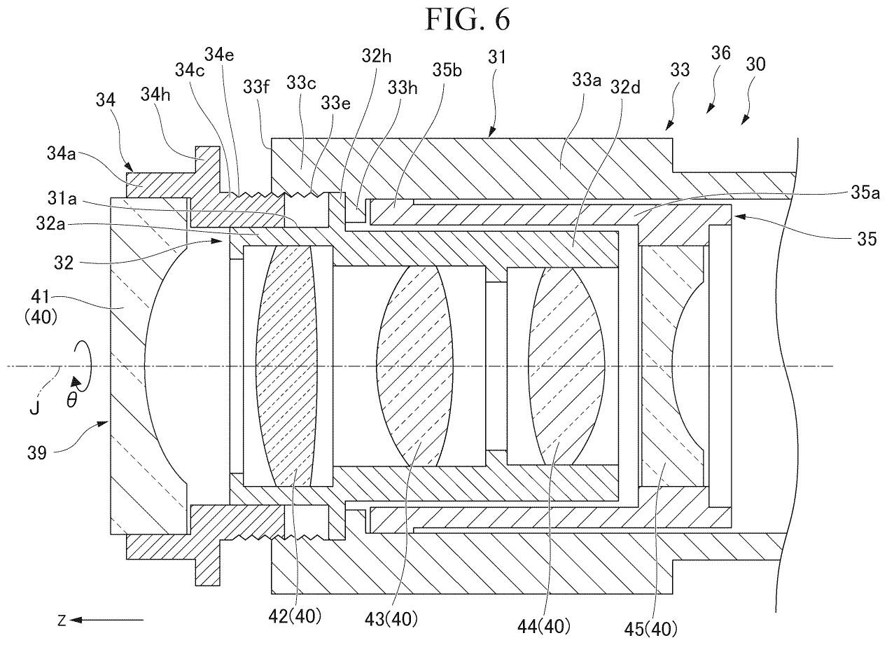

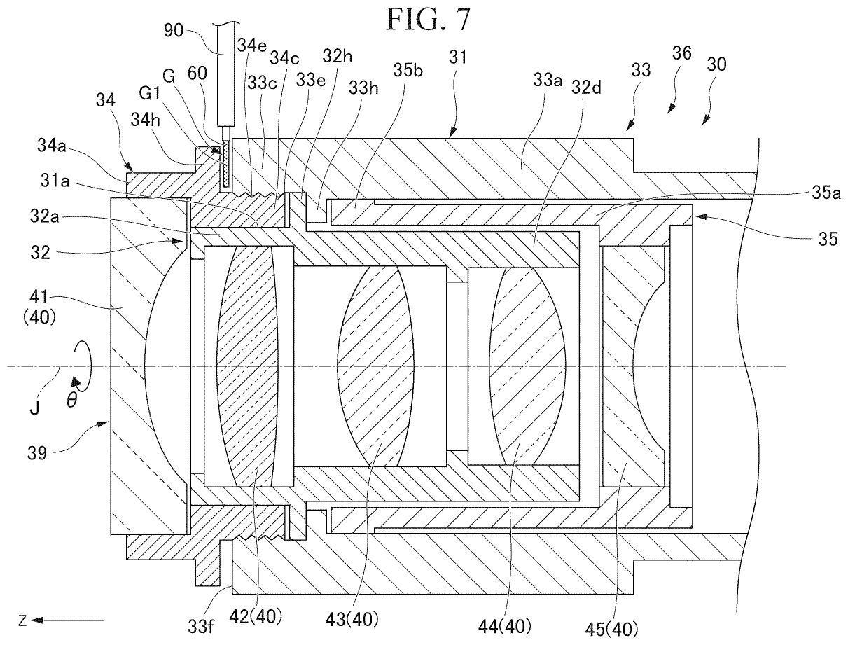

FIG. 4 is a flowchart showing a method Ma of assembling the optical unit 30 of the present embodiment. FIG. 5 is a first longitudinal cross-sectional view showing the method Ma of assembling the optical unit 30 of the present embodiment. FIG. 6 is a second longitudinal cross-sectional view showing the method Ma of assembling the optical unit 30 of the present embodiment. FIG. 7 is a third longitudinal cross-sectional view showing the method Ma of assembling the optical unit 30 of the present embodiment.

As shown in FIG. 4, the method Ma of assembling the optical unit 30 of the present embodiment includes an attachment process S01 of attaching the moving frame 35 and the first holding frame 32 to the second holding frame 33, an adjustment process S02 of adjusting the position of the first lens 41 held by the first lens frame 34, and a fixing process S03 of fixing the first lens frame 34 to the holding frame 31. In the following description, “an operator and the like” include an operator and an assembly device performing the operation of each process. The operation of each process may be performed by only the operator, only the assembly device, or both the operator and the assembly device.

In the attachment process S01, the first holding frame 32 and the moving frame 35 are attached to the second holding frame 33. As shown in FIG. 5, first, the operator and the like fix the second lens 42 to the first tubular portion 32a of the first holding frame 32 and fix the third lens 43 and the fourth lens 44 to the second tubular portion 32d. Next, the operator and the like move the first holding frame 32 from the object side (+Z side) of the second holding frame 33 fixed to a jig or the like to the image side (−Z side) and insert the first holding frame 32 into the second holding frame 33. As shown in FIG. 6, the operator and the like stop moving of the first holding frame 32 when the first holding frame 32 moves to the image side until the first annular portion 32h contacts the second annular portion 33h in the optical axis direction. At this time, as described above, the first annular portion 32h is fitted to the portion of the inner circumferential surface of the fourth tubular portion 33c on the image side, and thus the first holding frame 32 is attached to the second holding frame 33. In addition, when the first holding frame 32 is attached to the second holding frame 33, the recess 31a is formed.

Next, the operator and the like fix the fifth lens 45 to the moving main body portion 35a of the moving frame 35 as shown in FIG. 5. Next, the operator and the like move the moving frame 35 from the image side (−Z side) of the second holding frame 33 to the object side (+Z side) and inserts the moving frame 35 into the second holding frame 33. As shown in FIG. 6, the operator and the like stop moving of the moving frame 35 when the position of the moving frame 35 relative to the second holding frame 33 in the optical axis direction reaches a predetermined position. At this time, the moving protrusion portion 35b contacts the inner circumferential surface of the third tubular portion 33a in the radial direction, and thus the moving frame 35 is attached to the second holding frame 33. When the first holding frame 32 and the moving frame 35 are attached to the second holding frame 33, the attachment process S01 ends.

In the adjustment process S02, the position of the first lens 41 held by the first lens frame 34 is adjusted. More specifically, in the adjustment process S02, the angle of view of the optical system 39 is adjusted by adjusting the position of the first lens 41 in the optical axis direction relative to the other lenses 42 to 45. First, the operator and the like fix the first lens 41 to the holding portion 34a of the first lens frame 34. Next, the operator and the like rotate the first lens frame 34 in the circumferential direction to screw the first screw portion 34e into the second screw portion 33e. As a result, the first lens frame 34 moves to the image side (−Z side) relative to the holding frame 31, and thus the first lens 41 moves to the image side relative to the other lenses 42 to 45. In addition, the protrusion portion 34c is inserted into the recess 31a. The operator and the like check the angle of view of the optical system 39 while screwing the first screw portion 34e into the second screw portion 33e, and ends rotation of the first lens frame 34 in the circumferential direction when the angle of view of the optical system 39 reaches a desired angle of view. That is, the operator and the like adjust the angle of view of the optical system 39 by adjusting the position of the first lens 41 while screwing the first screw portion 34e into the second screw portion 33e. Therefore, in the adjustment process S02 of the present embodiment, the position of the first lens 41 can be easily adjusted by adjusting the amount of screwing of the first screw portion 34e into the second screw portion 33e. When adjustment of the position of the first lens 41 ends, the adjustment process S02 ends.

In the fixing process S03, the first lens frame 34 is fixed to the holding frame 31. In the present embodiment, the first lens frame 34 is fixed to the holding frame 31 after the position of the first lens 41 is adjusted in the adjustment process S02. As described above, in the present embodiment, the holding frame 31 and the first lens frame 34 are bonded and fixed by the adhesive 60. As shown in FIG. 7, the operator and the like inject the liquid adhesive 60 into the first gap G1 using a filling machine 90. Although not shown, the liquid adhesive 60 injected into the first gap G1 flows into the second gap G2, the third gap G3, and the fourth gap G4 shown in FIG. 3. As a result, the liquid adhesive 60 is filled in the entire gap G. When the liquid adhesive 60 is filled in the entire gap G, the operator and the like stop injecting the liquid adhesive 60. Thereafter, when the operator and the like harden the liquid adhesive 60, the first lens frame 34 is fixed to the holding frame 31, and the fixing process S03 ends. When the fixing process S03 ends, the optical unit 30 shown in FIG. 2 is constructed.

According to the present embodiment, the optical unit 30 is provided at the tip of the endoscope 2 and includes the plurality of lenses 40 that constitute the optical system 39, the cylindrical first lens frame 34 that holds the first lens 41 disposed closest to the object side (+Z side) among the plurality of lenses 40 and has the first screw portion 34e formed thereon, and the cylindrical holding frame 31 having the second screw portion 33e formed thereon, and the first screw portion 34e and the second screw portion 33e are fitted to each other such that the first lens frame 34 is fixed to the holding frame 31. Accordingly, as shown in FIG. 3, the first screw portion 34e formed on the first outer surface 34d and the second screw portion 33e formed on the first inner surface 33d are screw-fitted to each other, and thus the second gap G2 between the first screw portion 34e and the second screw portion 33e in the gap G between the first lens frame 34 and the holding frame 31 can be made to have a sawtooth shape extending in the optical axis direction, as described above. As a result, as described above, compared to a case in which the first screw portion 34e and the second screw portion 33e are not formed on the first outer surface 34d and the first inner surface 33d respectively, the penetration path Ri, which is a path through which moisture outside the optical unit 30 penetrates into the optical unit 30, can be made longer. Accordingly, it is possible to curb moisture outside the optical unit 30 from penetrating into the optical unit 30. Therefore, it is possible to curb condensation on the surface of the first lens 41 due to moisture inside the optical unit 30, and thus it is possible to curb a decrease in the image quality of optical images formed by the optical unit 30.

In addition, in the present embodiment, as described above, the position of the first lens 41 in the optical axis direction can be easily adjusted by adjusting the amount of screwing of the first screw portion 34e into the second screw portion 33e in the adjustment process S02. Therefore, the number of operations in the adjustment process S02 can be curbed from increasing, and thus it is possible to curb an increase in the man-hour for assembling the optical unit 30.

According to the present embodiment, the adhesive 60 is filled between the first screw portion 34e and the second screw portion 33e. Accordingly, the second gap G2 can be sealed by the adhesive 60, and thus penetration of moisture outside the optical unit 30 into the optical unit 30 can be more suitably curbed. Therefore, condensation on the surface of the first lens 41 can be more suitably curbed, and thus deterioration in the image quality of optical images formed by the optical unit 30 can be more suitably curbed.

In addition, in the present embodiment, the first screw portion 34e and the second screw portion 33e can be fixed by the adhesive 60, and thus the first lens frame 34 can be firmly fixed to the holding frame 31. This makes it possible to curb shifting of the position of the first lens 41 in the optical axis direction relative to the other lenses 42 to 45. Therefore, it is possible to improve the stability of the angle of view of the optical system 39, and thus improve the stability of the image quality of optical images formed by the optical unit 30.

According to the present embodiment, the first lens frame 34 is disposed closer to the object side (+Z side) than the first screw portion 34e and has the flange portion 34h that protrudes outward in the radial direction, the flange portion 34h faces the first opposing surface 33f, which is the surface of the outer surface of the holding frame 31 that faces the object side, and the adhesive 60 is filled between the flange portion 34h and the first opposing surface 33f. Therefore, since the first gap G1 can be sealed by the adhesive 60, it is possible to more suitably curb penetration of moisture from outside the optical unit 30 into the optical unit 30.

In addition, in the present embodiment, the adhesive 60 is filled in the entire gap G between the first lens frame 34 and the holding frame 31. Therefore, the entire gap G can be sealed by the adhesive 60, and thus penetration of moisture from outside the optical unit 30 into the optical unit 30 can be more suitably curbed.

In addition, in the present embodiment, the adhesive 60 is filled in the entire gap G as described above, and thus the first lens frame 34 can be more firmly fixed to the holding frame 31. Accordingly, it is possible to more suitably curb shifting of the position of the first lens 41 in the optical axis direction relative to the other lenses 42 to 45. Therefore, the stability of the angle of view of the optical system 39 can be more suitably improved, and thus the stability of the image quality of optical images formed by the optical unit 30 can be more suitably improved.

According to the present embodiment, the first screw portion 34e is formed on the outer circumferential surface of the first lens frame 34, and the second screw portion 33e is formed on the inner circumferential surface of the holding frame 31. Accordingly, it is possible to adjust the position of the first lens frame 34 in the optical axis direction relative to the holding frame 31 by adjusting the amount of screwing of the first screw portion 34e into the second screw portion 33e in the adjustment process S02. Therefore, it is possible to easily adjust the position of the first lens 41 in the optical axis direction relative to the other lenses 42 to 45 by adjusting the amount of screwing, as described above. This makes it possible to improve the workability of the adjustment process S02, thereby curbing an increase in the man-hour for assembling the optical unit 30.

According to the present embodiment, the recess 31a recessed to the image side (−Z side) and extending around the circumference is provided in the holding frame 31, the first lens frame 34 has the cylindrical protrusion portion 34c protruding to the image side from the flange portion 34h and disposed inside the recess 31a, the first screw portion 34e is formed on the outer circumferential surface of the protrusion portion 34c, the second screw portion 33e is provided on a surface of the recess 31a facing inward in the radial direction, and the surface of the protrusion portion 34c facing inward in the radial direction faces the surface of the recess 31a facing outward in the radial direction in the radial direction. Therefore, as shown in FIG. 3, in the gap G, the third gap G3 and the fourth gap G4 extending in mutually orthogonal directions can be formed between the first screw portion 34e and the second screw portion 33e and the inside of the optical unit 30. Accordingly, the angle of the portion where the third gap G3 and the fourth gap G4 are connected in the gap G can be set to approximately 90°. Therefore, foreign matters such as cutting chips generated at the time of forming the first screw portion 34e and the second screw portion 33e, and wear powder generated at the time of screwing the first screw portion 34e into the second screw portion 33e can be easily curbed from entering the fourth gap G4 from the third gap G3. Therefore, such foreign matters can be easily curbed from entering the inside of the optical unit 30 through the third gap G3 and the fourth gap G4. This can curb such foreign matters from adhering to the surface of each lens and can curb defects such as scratches from occurring on the surface of each lens due to such foreign matters. Therefore, the stability of the image quality of optical images formed by the optical unit 30 can be more suitably improved.

According to the present embodiment, the holding frame 31 holds at least one of the lenses 42, 43, 44, and 45 which are disposed closer to the image side (−Z side) than the first lens 41 among the plurality of lenses 40. Accordingly, at least one of the lenses 42, 43, 44, and 45 is held by the holding frame 31 which is fixed to the first lens frame 34 which holds the first lens 41, making it easier to stabilize the position of the first lens 41 relative to the other lenses 42 to 45. This makes it possible to more suitably improve the stability of the angle of view of the optical system 39. Therefore, it is possible to more suitably improve the stability of the image quality of optical images formed by the optical unit 30.

According to the present embodiment, the endoscope 2 has the optical unit 30 provided at the tip thereof. As described above, the penetration path Ri, which is a path through which moisture from outside the optical unit 30 penetrates into the optical unit 30, can be lengthened. This makes it possible to curb penetration of moisture from the outside of the optical unit 30 into the optical unit 30, and thus it is possible to curb condensation on the surface of the first lens 41. Therefore, it is possible to curb a decrease in the image quality of optical images captured by the endoscope 2.

According to the present embodiment, the method Ma of assembling the optical unit 30 includes the adjustment process S02 of adjusting the position of the first lens 41 held by the first lens frame 34, and the fixing process S03 of fixing the first lens frame 34 to the holding frame 31, and fixes the first lens frame 34 to the holding frame 31 after adjusting the position of the first lens 41 while screwing the first screw portion 34e formed on the first lens frame 34 into the second screw portion 33e formed on the holding frame 31. Accordingly, as described above, the position of the first lens 41 can be easily adjusted by adjusting the amount of screwing of the first screw portion 34e into the second screw portion 33e in the adjustment process S02. Therefore, the operation of adjusting the position of the first lens 41 can be simplified compared to a case in which the position of the first lens 41 is adjusted by, for example, a jig that holds the first lens frame 34 and determines the position of the first lens frame 34 relative to the holding frame 31, in the adjustment process S02. Therefore, it is possible to curb an increase in the man-hour of the adjustment process S02, and thus curb an increase in the manufacturing cost and the man-hour for assembling the optical unit 30.

### Second Embodiment

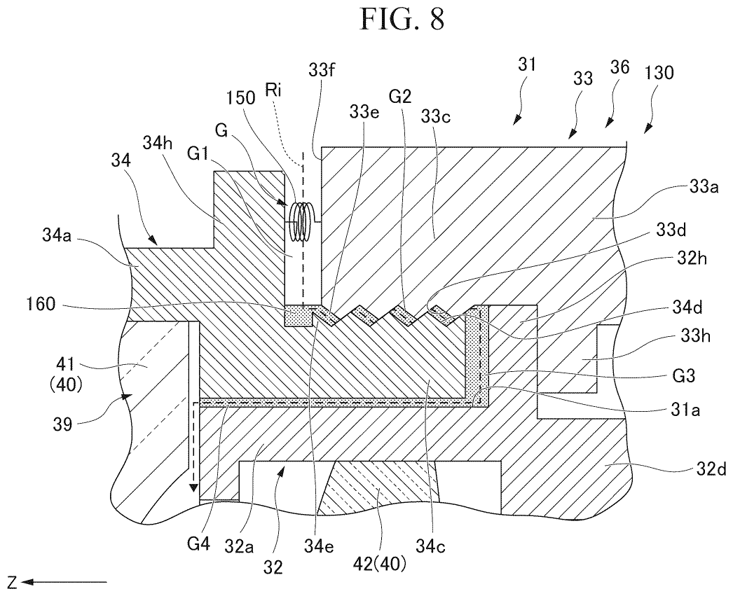

FIG. 8 is an enlarged longitudinal cross-sectional view showing an optical unit 130 of the present embodiment. In the following description, the same components as those in the first embodiment described above are denoted by the same reference numerals, and the description thereof will be omitted. As shown in FIG. 8, in the present embodiment, the optical unit 130 has an elastic member 150.

The elastic member 150 is disposed between the flange portion 34h and the first opposing surface 33f. In the present embodiment, the elastic member 150 is a spring member. The elastic member 150 may be a coil spring or a leaf spring. One end of the elastic member 150 is held by the first opposing surface 33f. The other end of the elastic member 150 is held by the surface of the flange portion 34h facing the image side (−Z side). The elastic member 150 is elastically deformable in the optical axis direction. The length of the elastic member 150 in the optical axis direction is less than the natural length of the elastic member 150. Therefore, the elastic member 150 presses the flange portion 34h toward the object side (+Z side). In other words, the elastic member 150 presses the first lens frame 34 toward the object side. Therefore, in the present embodiment, the surface of the first screw portion 34e facing in a direction inclined outward in the radial direction from the object side comes into contact with the surface of the second screw portion 33e facing in a direction inclined inward in the radial direction from the image side. This allows the first screw portion 34e and the second screw portion 33e to come into contact in the optical axis direction.

Although the adhesive 160 in the present embodiment is filled in the second gap G2, the third gap G3, and the fourth gap G4, the adhesive 160 is not filled in the first gap G1. The other configurations and the like of the optical unit 130 are the same as the other configurations and the like of the optical unit 30 in the first embodiment described above.

According to the present embodiment, the first lens frame 34 is disposed closer to the object side (+Z side) than the first screw portion 34e and has the flange portion 34h that protrudes outward in the radial direction, the flange portion 34h faces the first opposing surface 33f of the holding frame 31, and the elastic member 150 that presses the flange portion 34h toward the object side is disposed between the flange portion 34h and the first opposing surface 33f. Accordingly, as described above, the first screw portion 34e and the second screw portion 33e can be brought into contact with each other in the optical axis direction, and thus it is easy to curb moisture from passing in the optical axis direction in the second gap G2. Accordingly, moisture from outside the optical unit 130 can be more suitably curbed from penetrating into the optical unit 130. Therefore, condensation on the surface of the first lens 41 can be more suitably curbed, and thus deterioration in the image quality of optical images formed by the optical unit 130 can be more suitably curbed.

In addition, in the present embodiment, since the first lens frame 34 is pressed toward the object side (+Z side), the first screw portion 34e and the second screw portion 33e can stably come into contact with each other in the optical axis direction. This makes it easier to stabilize the position of the first lens frame 34 in the optical axis direction relative to the holding frame 31, and thus makes it easier to stabilize the position of the first lens 41 in the optical axis direction relative to the other lenses 42 to 45. Therefore, the stability of the image quality of optical images formed by the optical unit 130 can be more suitably improved.

According to the present embodiment, the elastic member 150 is a spring member. Accordingly, the elastic member 150 can be formed in a simple configuration, and thus an increase in the manufacturing cost of the elastic member 150 can be curbed. Therefore, an increase in the manufacturing cost of the optical unit 130 can be curbed.

### Modified Example of Second Embodiment

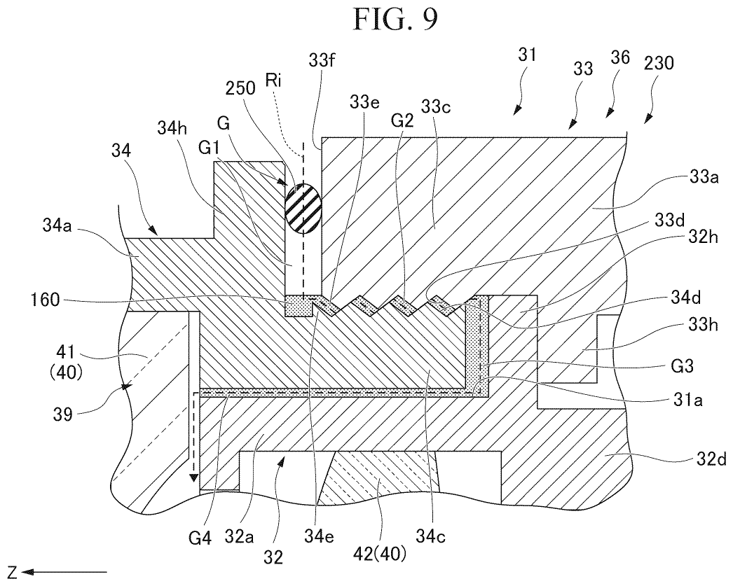

FIG. 9 is an enlarged longitudinal cross-sectional view showing an optical unit 230 of the present modification. In the following description, the same components as those of the second embodiment described above are denoted by the same reference numerals, and the description thereof will be omitted. As shown in FIG. 9, in the present modified example, the optical unit 230 has an elastic member 250.

The elastic member 250 is disposed between the flange portion 34h and the first opposing surface 33f. In the present modified example, the elastic member 250 is an O-ring. The elastic member 250 contacts the first opposing surface 33f and the surface of the flange portion 34h facing the image side (−Z side) in the optical axis direction. The elastic member 250 is elastically deformable in the optical axis direction. Accordingly, the elastic member 250 presses the flange portion 34h toward the object side (+Z side). In other words, the elastic member 250 presses the first lens frame 34 toward the object side. Therefore, in the present modified example, the first screw portion 34e and the second screw portion 33e can be brought into contact with each other in the optical axis direction as in the second embodiment described above. The other configurations and the like of the optical unit 230 are the same as the other configurations and the like of the optical unit 130 of the second embodiment described above.

According to the present modified example, the first lens frame 34 is disposed closer to the object side (+Z side) than the first screw portion 34e and has the flange portion 34h that protrudes outward in the radial direction, the flange portion 34h faces the first opposing surface 33f of the holding frame 31, and the elastic member 250 that presses the flange portion 34h toward the object side is disposed between the flange portion 34h and the first opposing surface 33f. Accordingly, as in the second embodiment described above, the first screw portion 34e and the second screw portion 33e can be brought into contact with each other in the optical axis direction, and thus moisture from outside the optical unit 230 can be more suitably curbed from penetrating into the optical unit 230. Therefore, condensation on the surface of the first lens 41 can be more suitably curbed, and deterioration in the image quality of optical images formed by the optical unit 230 can be more suitably curbed.

According to the present modified example, the elastic member 250 is an O-ring. Accordingly, the elastic member 250 can be formed in a simple configuration, and an increase in the manufacturing cost of the elastic member 250 can be curbed. Therefore, an increase in the manufacturing cost of the optical unit 230 can be curbed.

In addition, in the present modified example, the elastic member 250 can seal the gap between the first opposing surface 33f and the flange portion 34h. Therefore, it is possible to more suitably curb penetration of moisture from the outside of the optical unit 230 into the optical unit 230.

### Third Embodiment

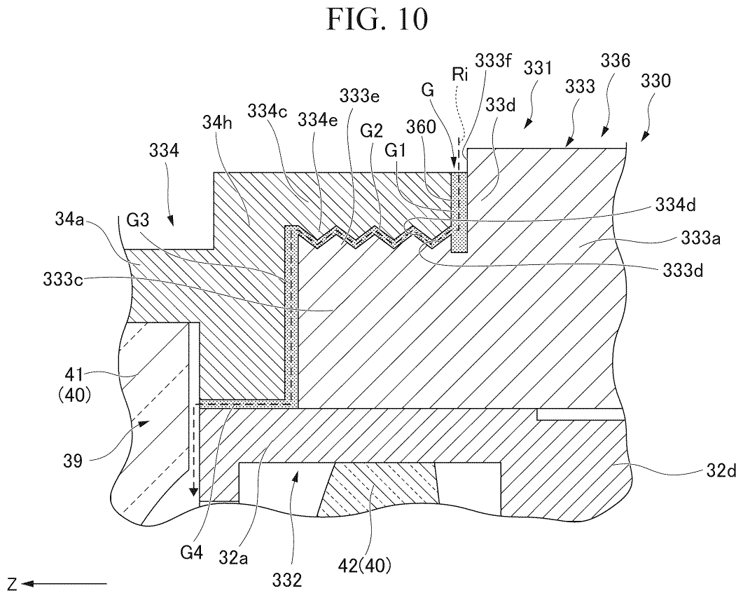

FIG. 10 is an enlarged longitudinal cross-sectional view showing an optical unit 330 of the present embodiment. In the following description, the same components as those in the first embodiment described above are denoted by the same reference numerals, and the description thereof will be omitted. As shown in FIG. 10, in the present embodiment, the optical unit 330 includes a housing 336, a plurality of lenses 40, and an adhesive 360. The housing 336 includes a holding frame 331, a first lens frame 334, and the moving frame 35 (refer to FIG. 2).

In the present embodiment, the holding frame 331 is cylindrical and extends in the optical axis direction. The holding frame 331 holds the first lens frame 334, the moving frame 35, and some of the plurality of lenses 40. The holding frame 331 includes a first holding frame 332 and a second holding frame 333.

The first holding frame 332 is substantially cylindrical and extends in the optical axis direction with the optical axis J as a center. The first holding frame 332 has a first tubular portion 32a and a second tubular portion 32d. The configurations and the like of the first tubular portion 32a and the second tubular portion 32d are the same as the configurations and the like of the first tubular portion 32a and the second tubular portion 32d in the first embodiment described above.

The second holding frame 333 holds the first holding frame 332, the first lens frame 334, and the moving frame 35. The second holding frame 333 is substantially cylindrical and extends in the optical axis direction with the optical axis J as a center. The second holding frame 333 surrounds the first holding frame 332 and the moving frame 35 from the outside in the radial direction. The second holding frame 333 has a third tubular portion 333a and a fourth tubular portion 333c.

The third tubular portion 333a is substantially cylindrical and extends in the optical axis direction with the optical axis J as a center. Although not shown, the end of the third tubular portion 333a on the image side (−Z side) is the end of the second holding frame 333 on the image side. The third tubular portion 333a surrounds the first holding frame 332 and the moving frame 35 from the outside in the radial direction. The end of the third tubular portion 333a on the object side (+Z side) is located closer to the image side than the end of the first tubular portion 32a on the object side. The first tubular portion 32a is fitted to the inner circumferential surface of the third tubular portion 333a. Accordingly, the second holding frame 333 holds the first holding frame 332. The third tubular portion 333a has a first opposing surface 333f.

The first opposing surface 333f is a surface of the outer surface of the third tubular portion 333a that faces the object side (+Z side). The first opposing surface 333f is located outward in the radial direction from the fourth tubular portion 333c. When viewed from the optical axis direction, the first opposing surface 333f is a substantially circular ring shape with the optical axis J as a center. The other configurations and the like of the third tubular portion 333a of the present embodiment are the same as the other configurations and the like of the third tubular portion 33a of the first embodiment described above.

The fourth tubular portion 333c is substantially cylindrical and protrudes in the optical axis direction with the optical axis J as a center. The end of the fourth tubular portion 333c on the object side (+Z side) is the end of the second holding frame 333 on the object side. The end of the fourth tubular portion 333c on the image side (−Z side) is connected to the third tubular portion 333a in the optical axis direction. The first tubular portion 32a is fitted to the inner circumferential surface of the fourth tubular portion 333c. Accordingly, the second holding frame 333 holds the first holding frame 332. The outer diameter of the fourth tubular portion 333c is less than the outer diameter of the third tubular portion 333a. The fourth tubular portion 333c has a second outer surface 333d. The second outer surface 333d is the outer circumferential surface of the fourth tubular portion 333c, that is, the surface facing outward in the radial direction.

A second screw portion 333e is formed on the fourth tubular portion 333c. That is, the second screw portion 333e is formed on the holding frame 331. The second screw portion 333e is a male screw formed on the second outer surface 333d. That is, the second screw portion 333e is formed on the outer circumferential surface of the holding frame 331. Other configurations and the like of the fourth tubular portion 333c of the present embodiment are the same as other configurations and the like of the fourth tubular portion 33c of the first embodiment described above.

The first lens frame 334 holds at least the first lens 41. In the present embodiment, the first lens frame 334 is substantially cylindrical with the optical axis J as a center. The first lens frame 334 has a holding portion 34a, a protrusion portion 334c, and a flange portion 34h. The configurations of the holding portion 34a and the flange portion 34h of the present embodiment are the same as those of the holding portion 34a and the flange portion 34h of the first embodiment described above.

The protrusion portion 334c is cylindrical and protrudes from the outer portion of the flange portion 34h in the radial direction to the image side (−Z side). In the present embodiment, the protrusion portion 334c is substantially cylindrical and has the optical axis J as a center. The protrusion portion 334c surrounds the fourth tubular portion 333c from the outer side in the radial direction. The protrusion portion 334c is disposed having a gap from the fourth tubular portion 333c in the radial direction. The protrusion portion 334c is disposed closer to the object side (+Z side) than the first opposing surface 333f. The protrusion portion 334c is disposed having a gap from the first opposing surface 333f in the optical axis direction. The protrusion portion 334c has a second inner surface 334d.

The second inner surface 334d is the inner circumferential surface of the protrusion portion 334c, that is, the surface facing inward in the radial direction. The second inner surface 334d faces the second outer surface 333d in the radial direction. The protrusion portion 334c has a first screw portion 334e formed thereon. That is, the first lens frame 334 has the first screw portion 334e formed thereon. In the present embodiment, the first screw portion 334e is formed on the second inner surface 334d. In the present embodiment, the first screw portion 334e is a female screw formed on the inner circumferential surface of the first lens frame 334. The first screw portion 334e is fitted to the second screw portion 333e. The first lens frame 334 is fixed to the holding frame 331 by fitting the first screw portion 334e and the second screw portion 333e to each other.

In the present embodiment, a gap G is provided between the holding frame 331 and the first lens frame 334. The gap G connects the outside of the optical unit 330 and the inside of the optical unit 330. The gap G includes a first gap G1, a second gap G2, a third gap G3, and a fourth gap G4. The first gap G1 is a gap between the first opposing surface 333f and the protrusion portion 334c. The first gap G1 connects the outside of the optical unit 330 and the second gap G2. The first gap G1 extends in the radial direction. The second gap G2 is a gap between the first screw portion 334e and the second screw portion 333e. The second gap G2 connects the first gap G1 and the third gap G3. In a longitudinal cross-sectional view, the second gap G2 has a sawtooth shape extending in the optical axis direction. Therefore, similarly to the first embodiment described above, the length of the second gap G2 can be increased compared to a case in which the first screw portion 334e and the second screw portion 333e are not provided on the second inner surface 334d and the second outer surface 333d respectively. This makes it possible to make the penetration path Ri longer.

The third gap G3 is a gap between the surface of the flange portion 34h facing the image side (−Z side) and the surface of the fourth tubular portion 333c facing the object side (+Z side). The third gap G3 connects the second gap G2 and the fourth gap G4. The third gap G3 extends in the radial direction. The fourth gap G4 is a gap between the surface of the flange portion 34h facing inward in the radial direction and the surface of the first tubular portion 32a facing outward in the radial direction. The fourth gap G4 connects the third gap G3 and the inside of the optical unit 330. The fourth gap G4 extends in the optical axis direction.

The adhesive 360 fixes the holding frame 331 and the first lens frame 334. The adhesive 360 is filled in the gap G. The adhesive 360 is filled in each of the first gap G1, the second gap G2, the third gap G3, and the fourth gap G4. Accordingly, the gap G between the holding frame 331 and the first lens frame 334 is sealed by the adhesive 360. Other configurations and the like of the adhesive 360 in the present embodiment are the same as other configurations and the like of the adhesive 60 in the first embodiment described above.

A method Ma of assembling the optical unit 30 in the present embodiment includes an attachment process S01, an adjustment process S02, and a fixing process S03. In the attachment process S01 in the present embodiment, the operator and the like insert the first holding frame 332 to which the second lens 42, the third lens 43 (refer to FIG. 5), and the fourth lens 44 (refer to FIG. 5) are fixed into the second holding frame 333, and fit the first tubular portion 32a to the third tubular portion 333a and the fourth tubular portion 333c. Accordingly, the first holding frame 332 is attached to the second holding frame 333. Other operations and the like in the attachment process S01 of the present embodiment are the same as the other operations and the like in the attachment process S01 of the first embodiment described above.

In the adjustment process S02 of the present embodiment, the operator and the like fix the first lens 41 to the holding portion 34a, and then rotate the first lens frame 334 in the circumferential direction to screw the first screw portion 334e into the second screw portion 333e. As in the first embodiment described above, the operator and the like check the angle of view of the optical system 39 while screwing the first screw portion 334e into the second screw portion 333e, and when the angle of view of the optical system 39 reaches a desired angle of view, ends the circumferential rotation of the first lens frame 334. Accordingly, the position of the first lens 41 is adjusted. When adjustment of the position of the first lens 41 is completed, the adjustment process S02 ends. The other operations and the like of the adjustment process S02 of the present embodiment are the same as the other operations and the like of the adjustment process S02 of the first embodiment described above.

In the fixing process S03 of the present embodiment, the operator and the like use the filling machine 90 to inject the liquid adhesive 360 into the first gap G1. Although not shown, the liquid adhesive 360 injected into the first gap G1 flows into the second gap G2, the third gap G3, and the fourth gap G4, and thus the entire gap G is filled with the liquid adhesive 360. Thereafter, when the operator and the like harden the liquid adhesive 360, the first lens frame 334 is fixed to the holding frame 331, and the fixing process S03 ends. The other operations and the like of the fixing process S03 of the present embodiment are the same as the other operations and the like of the fixing process S03 of the first embodiment described above.

According to the present embodiment, the first screw portion 334e is formed on the inner circumferential surface of the first lens frame 334, and the second screw portion 333e is formed on the outer circumferential surface of the holding frame 331. Therefore, as described above, the position of the first lens 41 in the optical axis direction relative to the other lenses 42 to 45 can be easily adjusted by adjusting the amount of screwing of the first screw portion 334e into the second screw portion 333e in the adjustment process S02. This improves the workability of the adjustment process S02, and thus it is possible to curb an increase in the man-hour for assembling the optical unit 330.

Furthermore, in the present embodiment, as described above, the first screw portion 334e and the second screw portion 333e are fitted to each other, and thus the first lens frame 334 is fixed to the holding frame 331. Therefore, as described above, the second gap G2 can be made to have a sawtooth shape extending in the optical axis direction, and the penetration path Ri can be lengthened. This can suitably curb penetration of moisture from outside the optical unit 330 into the optical unit 330. Therefore, it is possible to curb condensation on the surface of the first lens 41, and thus curb a decrease in the image quality of optical images formed by the optical unit 330.

Further, in the present embodiment, the second gap G2 between the first screw portion 334e and the second screw portion 333e is filled with the adhesive 360. Therefore, the second gap G2 can be sealed by the adhesive 360, and thus moisture from outside the optical unit 330 can be more suitably curbed from penetrating into the optical unit 330.

Furthermore, in the present embodiment, the entire gap G between the first lens frame 334 and the holding frame 331 is filled with the adhesive 360. Therefore, the entire gap G can be sealed by the adhesive 360, and thus moisture from outside the optical unit 330 can be more suitably curbed from penetrating into the optical unit 330.

### Fourth Embodiment

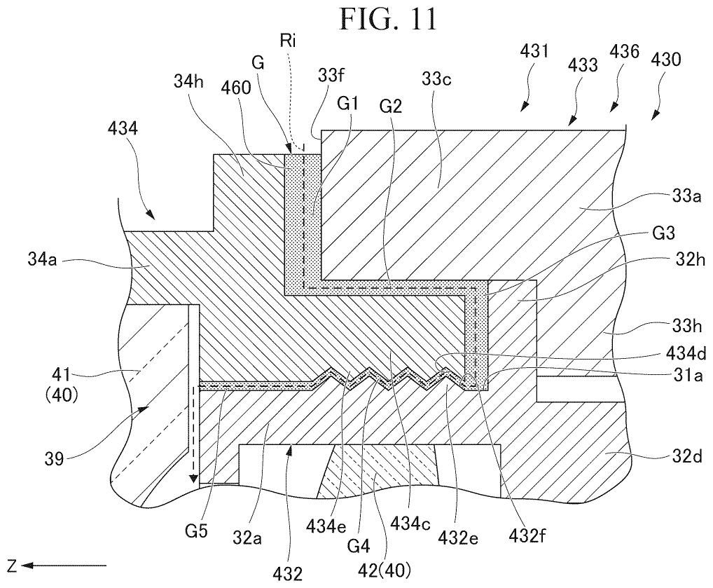

FIG. 11 is an enlarged longitudinal cross-sectional view showing an optical unit 430 of the present embodiment. In the following description, the same components as those of the first embodiment described above are denoted by the same reference numerals, and the description thereof will be omitted. As shown in FIG. 11, in the present embodiment, the optical unit 430 includes a housing 436, a plurality of lenses 40, and an adhesive 460. The housing 436 includes a holding frame 431, a first lens frame 434, and the moving frame 35 (refer to FIG. 2).

In the present embodiment, the holding frame 431 is cylindrical and extends in the optical axis direction. The holding frame 431 holds the first lens frame 434, the moving frame 35, and some of the lenses 40. The holding frame 431 includes a first holding frame 432 and a second holding frame 433. A recess 31a is provided in the holding frame 431.

The first holding frame 432 is substantially cylindrical and extends in the optical axis direction with the optical axis J as a center. As in the first embodiment described above, the first holding frame 432 holds at least one of the lenses 40 that is disposed closer to the image side (−Z side) than the first lens 41. The first holding frame 432 has a first tubular portion 32a, a second tubular portion 32d, and a first annular portion 32h. The first tubular portion 32a has a third outer surface 432f. The third outer surface 432f is the outer circumferential surface of the first tubular portion 32a, that is, the surface facing outward in the radial direction.

A second screw portion 432e is formed on the first tubular portion 32a. That is, in the present embodiment, the second screw portion 432e is formed in the first holding frame 432. That is, the second screw portion 432e is formed in the holding frame 431. The second screw portion 432e is a male screw formed on the portion of the third outer surface 432f on the image side (−Z side). That is, the second screw portion 432e is formed on the outer circumferential surface of the holding frame 431. In the optical axis direction, the second screw portion 432e is disposed between the flange portion 34h and the first annular portion 32h. The other configurations and the like of the first holding frame 432 of the present embodiment are the same as the other configurations and the like of the first holding frame 32 of the first embodiment described above.

The second holding frame 433 holds the first holding frame 432 and the moving frame 35. The second holding frame 433 is substantially cylindrical and extends in the optical axis direction with the optical axis J as a center. The second holding frame 433 surrounds the first holding frame 432 and the moving frame 35 from the outside in the radial direction. The second holding frame 433 has a third tubular portion 33a and a fourth tubular portion 33c. The fourth tubular portion 33c of the present embodiment does not have a second screw portion formed thereon. The other configurations and the like of the second holding frame 433 of the present embodiment are the same as the other configurations and the like of the second holding frame 33 of the first embodiment described above.

The first lens frame 434 holds at least the first lens 41. In the present embodiment, the first lens frame 434 is substantially cylindrical with the optical axis J as a center. The first lens frame 434 has a holding portion 34a, a protrusion portion 434c, and a flange portion 34h. The configurations of the holding portion 34a and the flange portion 34h of the present embodiment are the same as the configurations of the holding portion 34a and the flange portion 34h of the first embodiment described above.

The protrusion portion 434c is cylindrical and protrudes from the flange portion 34h to the image side (−Z side). In the present embodiment, the protrusion portion 434c is substantially cylindrical and has the optical axis J as a center. The inner diameter of the protrusion portion 434c is the same as the inner diameter of the flange portion 34h. The protrusion portion 434c surrounds the first tubular portion 32a from the outside in the radial direction. The protrusion portion 434c is disposed having a gap from the first tubular portion 32a in the radial direction. A portion of the protrusion portion 434c on the image side is disposed inside the recess 31a. That is, at least a part of the protrusion portion 434c is disposed inside the recess 31a. The protrusion portion 434c has a third inner surface 434d.

The third inner surface 434d is the inner circumferential surface of the protrusion portion 434c, that is, the surface facing inward in the radial direction. The third inner surface 434d faces the third outer surface 432f in the radial direction. A first screw portion 434e is formed on the protrusion portion 434c. That is, the first screw portion 434e is formed on the first lens frame 434. The first screw portion 434e is formed on the third inner surface 434d. In the present embodiment, the first screw portion 434e is a female screw formed on the inner circumferential surface of the first lens frame 434. The first screw portion 434e is fitted to the second screw portion 432e. The first lens frame 434 is fixed to the holding frame 431 by fitting the first screw portion 434e and the second screw portion 432e to each other.

In the present embodiment, a gap G is provided between the holding frame 431 and the first lens frame 434. The gap G connects the outside of the optical unit 430 and the inside of the optical unit 430. The gap G includes a first gap G1, a second gap G2, a third gap G3, a fourth gap G4, and a fifth gap G5. The first gap G1 is a gap between the first opposing surface 33f and the flange portion 34h. The first gap G1 connects the outside of the optical unit 430 and the second gap G2. The first gap G1 extends in the radial direction. The second gap G2 is a gap between the inner circumferential surface of the fourth tubular portion 33c and the outer circumferential surface of the protrusion portion 434c. The second gap G2 connects the first gap G1 and the third gap G3. The second gap G2 extends in the optical axis direction. The third gap G3 is a gap between the surface of the first annular portion 32h facing the object side (+Z side) and the surface of the protrusion portion 434c facing the image side (−Z side). The third gap G3 connects the second gap G2 and the fourth gap G4. The third gap G3 extends in the radial direction.

The fourth gap G4 is a gap between the first screw portion 434e and the second screw portion 432e. The fourth gap G4 connects the third gap G3 and the fifth gap G5. In a longitudinal cross-sectional view, the fourth gap G4 has a sawtooth shape extending in the optical axis direction. Accordingly, in the present embodiment, the length of the fourth gap G4 can be increased compared to a case in which the first screw portion 434e and the second screw portion 432e are not provided on the third inner surface 434d and the third outer surface 432f respectively. Therefore, the penetration path Ri can be increased. The fifth gap G5 is a gap between the portion of the third outer surface 432f on the object side (+Z side) and the surface of the flange portion 34h facing inward in the radial direction. The fifth gap G5 connects the fourth gap G4 and the inside of the optical unit 430. The fifth gap G5 extends in the optical axis direction.

The adhesive 460 fixes the holding frame 431 and the first lens frame 434. The adhesive 460 is filled in the gap G. The adhesive 460 is filled in each of the first gap G1, the second gap G2, the third gap G3, the fourth gap G4, and the fifth gap G5. Accordingly, the gap G between the holding frame 431 and the first lens frame 434 is sealed by the adhesive 460. Other configurations and the like of the adhesive 460 in the present embodiment are the same as other configurations and the like of the adhesive 60 in the first embodiment described above.

A method Ma of assembling the optical unit 30 in the present embodiment includes an attachment process S01, an adjustment process S02, and a fixing process S03. Operations and the like in the attachment process S01 in the present embodiment are the same as the operations and the like in the attachment process S01 in the first embodiment described above.

In the adjustment process S02 of the present embodiment, the operator and the like fix the first lens 41 to the holding portion 34a, and then rotate the first lens frame 434 in the circumferential direction to screw the first screw portion 434e into the second screw portion 432e. As in the first embodiment described above, the operator and the like check the angle of view of the optical system 39 while screwing the first screw portion 434e into the second screw portion 432e, and when the angle of view of the optical system 39 reaches a desired angle of view, ends the circumferential rotation of the first lens frame 434. Accordingly, the position of the first lens 41 is adjusted. When adjustment of the position of the first lens 41 is completed, the adjustment process S02 ends. The other operations and the like in the adjustment process S02 of the present embodiment are the same as the other operations and the like in the adjustment process S02 of the first embodiment described above.

In the fixing process S03 of the present embodiment, the operator and the like inject the liquid adhesive 460 into the first gap G1 using the filling machine 90. Although not shown, the liquid adhesive 460 injected into the first gap G1 flows into the second gap G2, the third gap G3, the fourth gap G4, and the fifth gap G5, and thus the entire gap G is filled with the liquid adhesive 460. Thereafter, when the operator and the like harden the liquid adhesive 460, the first lens frame 434 is fixed to the holding frame 431, and the fixing process S03 ends. The other operations and the like of the fixing process S03 of the present embodiment are the same as the other operations and the like of the fixing process S03 of the first embodiment described above.

According to the present embodiment, the holding frame 431 includes the first holding frame 432 that holds at least one of the lenses 40 that is disposed closer to the image side (−Z side) than the first lens 41, and the second screw portion 432e is formed on the first holding frame 432. Accordingly, since the first lens frame 434 that holds the first lens 41 is fixed to the first holding frame 432 that holds some of the lenses 40, it is easy to improve the accuracy of the position of the first lens 41 relative to the lenses held by the first holding frame 432. Therefore, it is possible to more suitably improve the stability of the image quality of optical images formed by the optical unit 430.

Further, in the present embodiment, as described above, the first screw portion 434e and the second screw portion 432e are fitted to each other, and thus the first lens frame 434 is fixed to the holding frame 431. Therefore, as described above, the fourth gap G4 can be formed in a sawtooth shape extending in the optical axis direction, and the penetration path Ri can be lengthened. This makes it possible to suitably curb penetration of moisture from the outside of the optical unit 430 into the optical unit 430.

In addition, in the present embodiment, the fourth gap G4, which is the gap between the first screw portion 434e and the second screw portion 432e, is filled with the adhesive 460. Therefore, the fourth gap G4 can be sealed by the adhesive 460, and thus it is possible to more suitably curb penetration of moisture from outside the optical unit 430 into the optical unit 430.

In addition, in the present embodiment, the entire gap G, which is the gap between the first lens frame 434 and the holding frame 431, is filled with the adhesive 460. Therefore, the entire gap G can be sealed by the adhesive 460, and thus it is possible to more suitably curb penetration of moisture from outside the optical unit 430 into the optical unit 430.

Furthermore, in the present embodiment, the first screw portion 434e is formed on the inner circumferential surface of the first lens frame 434, and the second screw portion 432e is formed on the outer circumferential surface of the holding frame 431. Therefore, as described above, the position of the first lens 41 can be adjusted by adjusting the amount of screwing of the first screw portion 434e into the second screw portion 432e in the adjustment process S02. This improves the workability of the adjustment process S02, and thus it is possible to curb an increase in the man-hour for assembling the optical unit 430.

Although the embodiments of the present invention have been described above, the configurations and combinations thereof in the embodiments are merely examples, and additions, omissions, substitutions, and other modifications of the configurations are possible without departing from the spirit of the present invention. Furthermore, the present invention is not limited to the embodiments.

The configuration of the housing is not limited to the present embodiment, and for example, the first and second holding frames may be integrally configured. That is, the first and second holding frames may be parts of the same single member. Further, the moving frame may be fixed to the holding frame.

The configuration of the adhesive is not limited to the present embodiment, and for example, the adhesive does not have to fill the entire gap. In this case, the adhesive does not have to fill between the first and second screw portions.

The method of assembling the optical unit is not limited to the present embodiment. For example, the operation procedure of the attachment process S01 is not limited to the present embodiment, and the first holding frame may be attached to the second holding frame after the moving frame is attached to the second holding frame, or the first holding frame and the moving frame may be attached to the second holding frame at the same time. Further, in a process prior to the attachment process, a liquid adhesive may be applied to the surfaces of the holding frame and the first lens frame. In this case, in the attachment process S01, when the first lens frame is attached to the holding frame, the liquid adhesive is filled in the gap. In this case, the liquid adhesive may not be injected into the gap in the fixing process.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. An optical unit provided at a tip of an endoscope, comprising:
a plurality of lenses constituting an optical system;
a cylindrical first lens frame holding at least a first lens disposed closest to an object side among the plurality of lenses and having a first screw portion formed on the first lens frame; and
a cylindrical holding frame on which a second screw portion is formed,
wherein the first screw portion and the second screw portion are fitted to each other such that the first lens frame is fixed to the holding frame.

2. The optical unit according to claim 1, wherein an adhesive is filled between the first screw portion and the second screw portion.

3. The optical unit according to claim 1, wherein the first lens frame is disposed closer to the object side than the first screw portion and has a flange portion protruding outward in a radial direction,
wherein the flange portion faces a first opposing surface that is a face in an outer surface of the holding frame which faces the object side, and the adhesive is filled between the flange portion and the first opposing surface.

4. The optical unit according to claim 1, wherein the first lens frame is disposed closer to the object side than the first screw portion and has a flange portion protruding outward in a radial direction,
wherein the flange portion faces a first opposing surface that is a surface in an outer surface of the holding frame which faces the object side, and an elastic member that presses the flange portion toward the object side is disposed between the flange portion and the first opposing surface.

5. The optical unit according to claim 4, wherein the elastic member is a spring member.

6. The optical unit according to claim 4, wherein the elastic member is an O-ring.

7. The optical unit according to claim 1, wherein the first screw portion is formed on an outer circumferential surface of the first lens frame, and the second screw portion is formed on an inner circumferential surface of the holding frame.

8. The optical unit according to claim 1, wherein the first screw portion is formed on an inner circumferential surface of the first lens frame, and the second screw portion is formed on an outer circumferential surface of the holding frame.

9. The optical unit according to claim 3, wherein the holding frame is provided with a recess recessed to an image side and extending around the circumference, the first lens frame has a cylindrical protrusion portion protruding from the flange portion to the image side, at least a part of the protrusion portion being disposed inside the recess, the first screw portion is formed on an outer circumferential surface of the protrusion portion, and the second screw portion is provided on a surface of the recess facing inward in the radial direction, and a surface of the protrusion portion facing inward in the radial direction faces a surface of the recess facing outward in the radial direction.

10. The optical unit according to claim 1, wherein the holding frame holds at least one of lenses disposed closer to the image side than the first lens among the plurality of lenses.

11. The optical unit according to claim 1, wherein the holding frame has a first holding frame holding at least one of lenses disposed closer to the image side than the first lens among the plurality of lenses, and
the second screw portion is formed on the first holding frame.

12. An endoscope having the optical unit according to claim 1 provided at a tip of the endoscope.

13. A method of assembling an optical unit to be provided at a tip of an endoscope, the method comprising:
an adjustment process of adjusting a position of a first lens disposed closest to an object side among a plurality of lenses constituting an optical system and held by a first lens frame; and
a fixing process of fixing the first lens frame to a holding frame,
wherein the position of the first lens is adjusted while a first screw portion formed on the first lens frame is screwed into a second screw portion formed on the holding frame, and then the first lens frame is fixed to the holding frame.
